(19) United States
(12) Plant Patent Application Publication
(10) Pub. No.: US 2017/0164540 P1
(43) Pub. Date: Jun. 8, 2017

(54) Anthurium plant named 'ANTHEMIEL'

(50) ;

(71) Applicant: ANTHURA B.V., Bleiswijk (NL)

(72) Inventor: Jan Van Dijk, Bleiswijk (NL)

(21) Appl. No.: 14/757,196

(22) Filed: Dec. 3, 2015

(51) Int. Cl. A01H 5/02

(52) U.S. Cl. PLT/368

(57) ABSTRACT

A new and distinct variety of Anthurium plant named ‘ANTHEMIEL’ particularly distinguished by a having small purple, heart-shaped and cupped spathe, a white spadix with yellow tip, a spathe that changes after 2 to 4 weeks from purple to red-purple and that retains this color for a long period, a compact plant form with a height of 18 cm to 20 cm and flowering continuously throughout the year, is disclosed.

Genus and species: Anthurium andreanum L.

Variety denomination: ‘ANTHEMIEL’.

### BACKGROUND OF THE NEW PLANT

The present invention comprises a new and distinct variety of Anthurium, botanically known as Anthurium andreanum L., and hereinafter referred to by the variety name ‘ANTHEMIEL’. The new variety originated from a hybridization made in October 2007 in Bleiswijk, The Netherlands. The female parent was a purple Anthurium pot plant designated ‘ANTHUCOEN’ (U.S. Plant Pat. No. 22,630), and the male parent was a red-purple Anthurium pot plant designated ‘11733-10’ (unpatented).

A single plant was selected in April 2009 and has been asexually reproduced repeatedly by tissue culture in Bleiswijk, The Netherlands over a six-year period. The present invention has been found to retain its distinctive characteristics through successive asexual propagations.

Plant Breeder's Rights for this variety have been applied for in the European Union on Nov. 18, 2013. ‘ANTHEMIEL’ has not been made publicly available or sold anywhere in the world more than one year prior to the filing of this application.

### SUMMARY OF THE INVENTION

The following are the most outstanding and distinguishing characteristics of this new variety when grown under normal horticultural practices in Bleiswijk, The Netherlands:
1) Very compact plant habit;
2) Small, red-purple, cordate spathe;
3) White spadix with yellow tip; and
4) Dark green, elliptical, cordate leaves.

### DESCRIPTION OF THE PHOTOGRAPHS

This new Anthurium plant is illustrated by the accompanying photographs which show the overall plant habit including blooms, buds and foliage of the plant; the colors shown are as true as can be reasonably obtained by conventional photographic procedures. The photographs are of a 35-week old plant grown in a greenhouse in Bleiswijk, The Netherlands in August 2015.

FIG. 1 shows the overall plant habit, including blooms, buds and foliage.

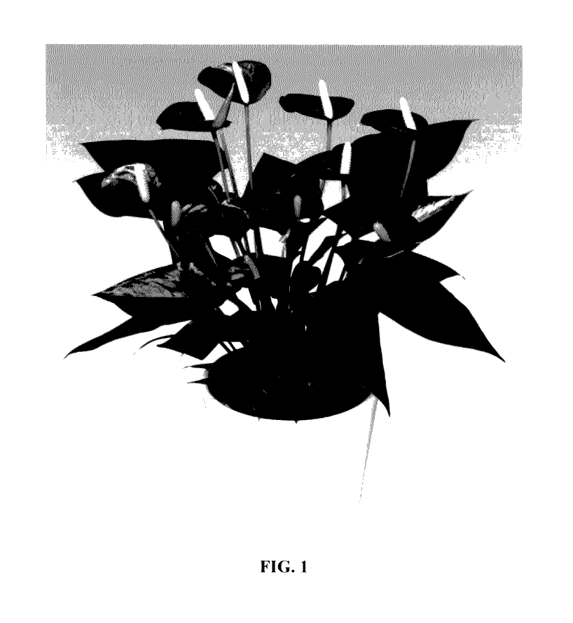

FIG. 2 shows a close-up of the mature spathe.

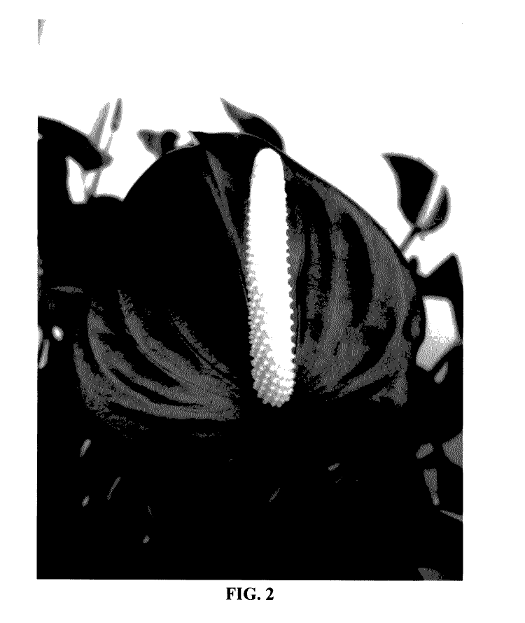

FIG. 3 shows the upper leaf blade surface.

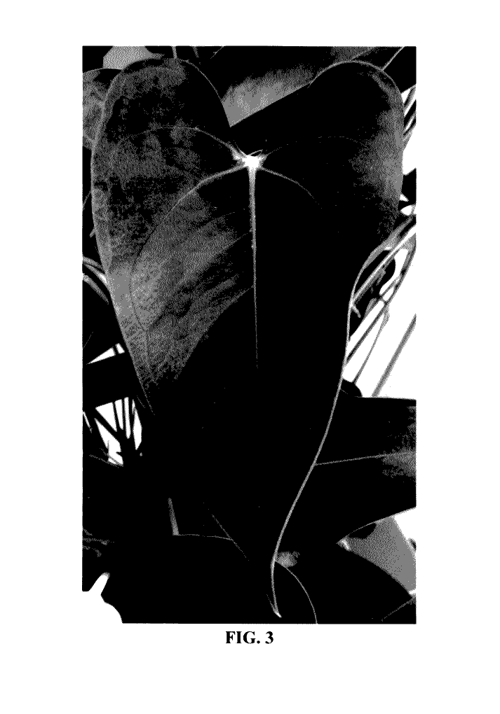

### DESCRIPTION OF THE NEW VARIETY

The following detailed description sets forth the distinctive characteristics of ‘ANTHEMIEL’. The data which define these characteristics were collected from asexual reproductions carried out in Bleiswijk, The Netherlands. The plant history was taken on 35-week old plants which were planted from tissue culture in 12-centimeter pots and grown in a glass greenhouse between 19° C. and 24° C. Observations were made in August 2015. Color readings were taken under 5000 lux natural light in the greenhouse. Color references are primarily to The R.H.S. Colour Chart of The Royal Horticultural Society of London (R.H.S.) (Sixth edition, 2015).

### DETAILED BOTANICAL DESCRIPTION

Classification:
   Family.—Araceae.
   Botanical.—Anthurium andreanum L.
   Common name.—Anthurium.
   Demonimation.—‘ANTHEMIEL’.
Parentage:
   Female parent.—Anthurium plant ‘ANTHUCOEN’ (U.S. Plant Pat. No. 22,630).
   Male parent.—Anthurium plant ‘11733-10’ (unpatented).
Plant:
   Propagation.—Tissue culture.
   Root description.—Fleshy-white colored roots with small hairy lateral roots having yellow-colored root tips.
   Time to produce a finished flowering plant.—28 to 33 weeks for a 12 cm pot.
   Growth habit.—Herbaceous perennial.
   Height (measured from soil, including inflorescence).—18.0 cm to 20.0 cm.
   Width (measured from leaf tips).—20.0 cm to 22.0 cm.
Leaves:
   Immature leaves.—Length: 7.0 cm to 9.0 cm Width: 4.0 cm to 5.0 cm Color: Upper surface: RHS 146A Lower surface: RHS 146C Texture (both upper and lower surfaces): Shiny.
   Mature leaves.—Length (fully expanded): 11.0 cm to 12.0 cm Width: 6.0 cm to 7.0 cm Shape: Elliptical cordate Apex: Acuminate Base: Cordate Leaf blade angle with the petiole: Between 120 degrees and 130 degrees Leaf margin: Entire Color: Upper surface: RHS NN137A Lower surface: RHS 144A Texture: Shiny, leathery and thick Venation: Pinnate veining; the mid-vein and primary veins (the veins that radiate out from the junction of petiole and leaf) protrude at the underside of the leaf blade Venation color: Upper surface: RHS 144A Lower surface: RHS 144C.
   Lobes.—Arrangement: Leaf blade has two lobes extending past the petiole. The lobes are non-touching Length of lobes of mature leaf blades: 1.5 cm to 2.5 cm Width of lobes of mature leaf blades: 2.5 cm to 3.5 cm Distance for petiole/leaf junction to highest point on lobes of mature leaf blades: 1.0 cm to 2.0 cm.
   Petiole.—Cross-section: Round Diameter: 0.2 cm to 0.25 cm Length: 8.0 cm to 10.0 cm for a mature leaf size Color: Mature leaf: RHS 144B Immature leaf: RHS 144B Cataphyll color surrounding the petiole: Outside: RHS 144C Inside: RHS 145B-D.
   Geniculum.—Length: 1.4 cm to 1.6 cm Width: 0.28 cm to 0.32 cm Color: RHS 144B.
Inflorescence:
   Arrangement.—Single.
   Flowering habit (length of flowering season).—Continuous.
   Number of inflorescences per plant.—6 to 8.
   Fragrance.—Absent.
   Longevity of inflorescence on plant.—Over a year.
Spathe:
   Buds.—The spathe is tightly rolled around the spadix and extrudes from the peduncle sheath. After the spathe is fully open the peduncle elongates some extra centimeters.
   Arrangement.—Spathe angle with the peduncle is between 100 degrees and 120 degrees; the spathe stands on a wiry peduncle about 3.0 cm to 5.0 cm above the foliage.
   Shape.—Cordate.
   Apex.—Acuminate.
   Base.—Cordate.
   Texture.—Shiny and slightly blistered.
   Margin.—Entire.
   Size.—Length: 3.0 cm to 4.5 cm Width: 2.8 cm to 4.3 cm.
   Lobes.—Arrangement: The spathe has two lobes extending past the peduncle. The lobes are non-touching Length: 0.4 cm to 0.8 cm Width: 1.5 cm to 2.0 cm.
   Color.—Just fully open: Upper surface: RHS 64A Lower surface: RHS 70B After 2 to 4 weeks: Upper surface: RHS 60A Lower surface: RHS 70C This red purple color remains for a very long period, at least more than 30 weeks after opening.
Peduncle:
   Shape.—Erect.
   Cross-section.—Round.
   Length.—10.0 cm to 15.0 cm.
   Diameter.—0.2 cm to 0.25 cm.
   Color.—RHS 144C.
Flowering time:
   General.—One small rooted untreated tissue culture plant of 6.0 cm tall will flower after 7 to 8 months, depending on the season, and 4 to 6 blossoms appear. More blossoms appear after some additional weeks so that a full flowering and commercial plant will have 6 to 8 red spathes. Smaller blossoms may occur on less mature plants.
Spadix:
   Size.—Length: 2.0 cm to 3.5 cm (depending on flower size) Width (at apex): 0.4 cm to 0.5 cm Width (at base): 0.6 cm to 0.7 cm.
   Shape.—Columnar.
   Angle from spadix tip to peduncle.—150 to 170 degrees.
   Texture.—When the spathe is unfurling the spadix is smooth. When the spadix matures, small stigmata protrude. The stigmata are evenly distributed round the spadix. The spadix matures from base to top, slowly giving the spadix a somewhat rough appearance.
   Color.—Immature: RHS 16B Mature: RHS 155B Aged: RHS 144B.
Flowers:
   Quantity per spadix.—200 to 250.
   Spadix flower arrangement.—Bisexual, rounded in cross-section.
   Shape.—Rounded.
   Size.—Length: 0.05 cm to 0.1 cm Diameter (maximum): 0.1 cm.
   Color.—RHS 155B.
Reproductive organs:
   Stamens.—Not visible.
   Pollen amount.—Very few.
   Pollen color.—RHS 156D.
   Pistil.—Quantity: Many Length: Less than 0.01 cm Color: RHS 156D.
   Style.—Not observed.
   Stigma.—Shape: Ovoid Diameter: Less than 0.01 cm Color: RHS 155B.
   Ovary.—Rarely visible.
   Ovary color.—Not measured.
Fruit and seed set: None observed.

### COMPARISON WITH PARENTAL AND COMMERCIAL VARIETIES

‘ANTHEMIEL’ differs from the female parent plant ‘ANTHUCOEN’ (U.S. Plant Pat. No. 22,630) in that ‘ANTHEMIEL’ has a white spadix with a yellow tip, whereas ‘ANTHUCOEN’ has a purple spadix.

‘ANTHEMIEL’ differs from male parent plant ‘11733-10’ (unpatented) in that ‘ANTHEMIEL’ has a narrower spathe width than ‘11733-10’.

‘ANTHEMIEL’ differs from commercial variety ‘ANTHDUBAQ’ (unpatented) in that ‘ANTHEMIEL’ has a white spadix with a yellow tip, whereas ‘ANTHDUBAQ’ has a purple spadix. Additionally, ‘ANTHEMIEL’ has a narrower spathe width than ‘ANTHDUBAQ’.

‘ANTHEMIEL’ differs from commercial variety ‘ANTHCOMEOR’ (unpatented) in that ‘ANTHEMIEL’ has a shorter plant height and a narrower spathe width than ‘ANTHCOMEOR’.

I claim:

1. A new and distinct variety of Anthurium plant named ‘ANTHEMIEL’, substantially as illustrated and described herein.
